(19) United States
(12) Patent Application Publication
(10) Pub. No.: US 2021/0386321 A1
(43) Pub. Date: Dec. 16, 2021

(54) DIAGNOSING PARTIAL OBSTRUCTIONS TO QUANTIFY THE BREATH DYNAMICS

(71) Applicant: Bat-Call Ltd., Nesher (IL)

(72) Inventors: Amir Landesberg, Haifa (IL); Jimy Pesin, Haifa (IL); Isak Gath, Kiryat Tivon (IL)

(73) Assignee: Bat-Call Ltd., Nesher (IL)

(21) Appl. No.: 17/283,559

(22) PCT Filed: Oct. 8, 2019
(86) PCT No.: PCT/IB2019/058538

§ 371 Date: Apr. 8, 2021

Related U.S. Application Data
(60) Provisional application No. 62/743,640, filed on Oct. 10, 2018.

(51) Int. Cl. A61B 5/08; A61B 5/085; A61B 5/113

(52) U.S. Cl. CPC A61B 5/0826; A61B 2562/0219; A61B 5/113; A61B 5/085

(57) ABSTRACT

Solutions are provided for immediate and precise diagnosis of partial obstruction in children and adults and for detection of potentially preventable events of accidental suffocation and strangulation and for the diagnosis of high upper airway resistance syndrome (UARS) or partial airway obstruction during sleep in adults. The solutions identify pathognomonic indices for partial obstruction by utilizing noninvasive miniature sensors for monitoring the breath dynamics.

### FIELD OF THE INVENTION

The present invention relates generally to diagnosis of partial obstruction in persons, and to detection of potentially preventable events of accidental suffocation and strangulation.

### BACKGROUND OF THE INVENTION

Accurate detection of partial and full obstructions is of great clinical benefit in children and adults. The rate of accidental suffocation and strangulation in bed (ASSB) in infants, many of which are potentially preventable, is about 12.5 deaths per 100,000 live births [1]. Sudden infant death syndrome (SIDS) is one of the leading causes of death in infants, with about 60 deaths per 100,000 births [2]. Interestingly, there is evidence that infants who died of SIDS had significantly more frequent episodes of obstructive sleep apnea [3]. The use of home apnea monitors for preventing SIDS is discouraged, as they have not been shown to be sufficiently effective [2, 4]. Numerous apnea monitoring methodologies primarily focus on central apnea events, but the ability to detect partial obstructions and hypoventilation has not been well established [5, 6].

Adult and Children with upper airway resistance syndrome (UARS) or obstructive sleep apnea syndrome suffer from frequent episodes of partial obstruction and have poor quality of life with daytime sleepiness and morbidities due to cardiovascular diseases [7, 8]. Interestingly, UARS without complete apnea causes morbidity similar to that observed with full obstructive apneas, including sleep fragmentation and daytime symptoms [8]. Episodes of partial obstructive or UARS are more difficult to detect than full-blown apneas [8].

Polysomnography is considered the gold standard for identifying and assessing the severity of obstructive sleep apnea, by measuring thoraco-abdominal dynamics in conjunction with airflow [9-11]. Plethysmography requires two belts on the chest and abdomen, which is poorly tolerated [6]. While thoraco-abdominal asynchrony is expected during obstructive episodes in adults [9], neonates and infants may normally exhibit this type of breathing [11, 12]. In parallel to the reported modest success of polysomnography-detected obstructive sleep apnea [9, 13-15], other published works have demonstrated inaccurate detection in infant [5, 12, 16], with low specificity of 10.9% [12], and even incidence of false negatives [16, 17]. Snore measurement has been shown to correlate well with the results of polysomnography in adults [18, 19]. However, while snoring is typically the first symptom of obstructive sleep apnea in adults, it does not always occur [20].

### SUMMARY OF THE INVENTION

The present invention seeks to address the unmet need of the art, that is, to accurately detect partial and full obstructive events, and to monitor and identify obstructions utilizing miniature accelerometers, as described more in detail below.

The present invention seeks to provide solutions for immediate and precise diagnosis of partial obstruction in children and adults, and for detection of the potentially preventable events of accidental suffocation and strangulation in bed, which are lacking in the prior art. The invention identifies pathognomonic indices for partial obstruction by utilizing miniature motion sensors, such as accelerometers, to monitor the breath dynamics.

The identification process includes identification of an increase in respiratory effort and identification of a change in breath signal shape (or simply breath shape) because of the obstruction (e.g., signals which are more rectangular) and/or phase difference between the chest and abdomen movement (that is, change in chest-abdominal movement synchrony).

Experiments were performed to carry out the invention. Six New Zealand rabbits were monitored during spontaneous breathing. Respiratory effort was determined from the esophageal pressure. Fully obstructive apneas, moderate and mild partial obstructions, three degrees of hypoxia (16%, 14%, and 12% FiO₂—fraction of inspired oxygen), and central apnea were induced. Breath dynamics were measured by accelerometers.

Energy, breath-shape, and chest-abdominal synchrony were extracted from the breath dynamics. Statistically significant changes were observed in all indices during partial obstruction. The energy correctly classified all the events as either increased (obstruction and hypoxia) or decreased (central apneas) respiratory effort. Subsequently, the elevated effort events were 100% correctly differentiated between partial obstruction and hypoxia. Changes in breath shape and chest-abdominal synchrony were significant even during mild partial obstructions, but unaltered by hypoxia, making them instrumental in classifying an obstruction.

Indices obtained from breath dynamics provide a novel and sensitive means of identifying partial obstructions and the specificity to distinguish them from non-obstructive elevations in respiratory effort.

### BRIEF DESCRIPTION OF THE DRAWINGS

The present invention will be understood and appreciated more fully from the following detailed description taken in conjunction with the drawings in which:

FIG. 1. Rabbit vital signs measured during one experiment containing all eight events (from left to right): three hypoxic events (FiO₂ of 16%, 14% and 12%), two full obstructions (Full Obst), two partial obstructions (Obst 50% and 25%), and one central-type apnea event.

FIG. 2. Five seconds of 10 Hz filtered data during (A) baseline, (B) 25% obstruction, (C) 16% hypoxia, and (D) central-type apnea. The top and bottom rows are from the right side of the chest and abdomen, respectively.

FIG. 3. The energy, entropy, shape-index (SI), phase-difference (PD) and respiratory rate (RR) during one experiment containing all eight events (from left to right): three hypoxic events (FiO₂ of 16%, 14% and 12%), two full obstructions (Full Obst), two partial obstructions (Obst 50% and 25%), and one central-type apnea event. Energy and entropy were used to detect changes in respiratory effort. The SI, PD and RR were used to differentiate between the obstructive and hypoxic events. SI and PD exhibited only minimal changes during hypoxia and significant changes during obstruction. Respiratory rate increased during hypoxic events and decreased during apneic events.

FIG. 4. Parameters used to detect changes in respiratory effort. Large increases from baseline energy (A) and entropy (B) were observed during hypoxia (Hyp) and obstruction (Obst), while both parameters decreased during central hypopnea\apnea. (* indicates p<0.05).

FIG. 5. The parameters used to classify obstructive and hypoxic events. The chest (A) and abdomen (B) shape-index (SI) were effectively the same at baseline and during hypoxia, and dramatically increased during obstruction. The phase difference (C) demonstrated no change during hypoxia, but a clear increase and positive phase during obstruction. (D) The respiratory rate (RR) increased during hypoxia, and decreased during obstruction. (* indicates p<0.05).

FIG. 6. K-means clustering using only two principal components in both classification stages yielded 100% correct classification. Energy and entropy were equally sensitive in successfully distinguishing between baseline and an increase in respiratory effort in the first stage of clustering. The combination of SI, chest-abdominal PD, and respiratory rate was sufficiently specific to differentiate between a mild obstruction and hypoxia event induced in the second stage of clustering. The X represents the centroids of that particular cluster.

### DETAILED DESCRIPTION OF EMBODIMENTS

The present invention provides solutions to accurately detect partial and full obstructive events, and to monitor and identify obstructions utilizing miniature accelerometers.

The miniature accelerometers were attached to both sides of the chest and at the epigastrium, and are used to monitor the breath dynamics [21-24]. This technology has been previously demonstrated to be effective in early detection of progressing pneumothorax in a pre-clinical study [21]. Moreover, in a clinical study in neonatal intensive care unit, the inventors have demonstrated that it can be used for early detection of hypoxemic episodes in ventilated infants during high-frequency oscillatory ventilation [22]. An editorial on the importance of early detection of deteriorating ventilation has highlighted the simplicity of this novel modality and the potential merits of monitoring the amplitude and symmetry of the ventilation [23]. Recently the inventors have shown that this modality enables immediate detection and classification of central and obstructive apneic episodes, with tight correlation with the esophageal-pressure (EP) [24]. The present study goals are to identify indices that tightly and specifically correlate with partial obstructions (in contrast to full obstructive apnea [24]), and to diminish false positives detection due to other forms of increased respiratory effort.

Methods

Experiments were performed with the approval of the Institutional Ethics Committee for the Care and Use of Animals. New Zealand white rabbits were anesthetized via an intramuscular injection of xylazine (5 mg/kg), ketamine (35 mg/kg), and acepromazine (1 mg/kg), followed by one-third of a dose every 45 minutes. The rabbits were tracheostomized and connected to a ventilator (SLE 2000, SLE, Surrey, UK), but were spontaneously breathing with a continuous positive airway pressure of 4 cmH₂O.

Two miniature (<1 g) accelerometers (Pneumonitor™, Yokneam, Israel) were attached to both sides of the chest, at the mid-clavicular lines and at the fifth or sixth intercostal space, and a third sensor was attached at the epigastrium, as previously described [21]. The heart rate, blood pressure (BP), oxygen saturation (SpO₂), end-tidal CO₂ (EtCO₂), and esophageal pressure (EP) were continuously acquired.

Methods

Four types of respiratory events were induced: fully obstructive apnea, partial obstructions (2 levels), hypoxic events (3 levels), and a central-type apnea. Fully obstructive apnea was created by completely clamping the endotracheal tube and was maintained for a maximum of 30 seconds, unless SpO₂<70% or hypotension (<40 mmHg) occurred first. The maximal EP during a full obstruction was used as an indicator of the maximal effort exerted by each animal. Partial occlusion was achieved by slowly tightening a clamp around the endotracheal tube until the EP rose to 50% or 25% of the maximal EP obtained during full obstruction. These events were denoted as 50% and 25% obstruction, respectively. Hypoxia was achieved by introducing nitrogen into the air mixture of the ventilator. Three levels of hypoxia were investigated: 16%, 14% and 12% FiO₂. The partial obstructions and the hypoxic event were maintained for 4 minutes. Pseudo-central-type apnea was induced at the end of the experiment by administration of succinylcholine (0.4 mg/kg) as previously described [25].

Analysis

The following parameters were extracted from the accelerometer signals: breath energy, breath entropy, breath shape index (SI), chest-abdominal phase difference (PD), and respiratory rate (RR) (See the supplementary data for more details). Energy and entropy were determined by averaging the breath signals collected from both the left and right sensors. The energy was used to quantify the intensity of the signal. The entropy is a measure of randomness in the signal and also accounts for breath-to-breath variability. The SI quantifies the smoothness of the breath motions, and is low for smooth sinusoidal waves and high for rectangular shaped and polyphasic signals. (A rectangular shaped signal is produced when the amplitude of the signal transitions from a minimum to a maximum value, dwells at the maximum value for some time, and then transitions back to the minimum value.) The SI was measured from the chest and abdomen. The PD is used to monitor asynchrony between the chest and abdomen as respiratory effort changes.

Principal component analysis was performed and the first two principal components, representing at least 80% of the variance in the data, were chosen. K-means clustering was implemented to separate event types into baseline, obstruction, and hypoxia. Additional detail is provided in the online data supplement.

Signal processing and statistical analysis were performed using Matlab (The MathWorks Inc., Natick, Mass., USA). Results are presented as mean±STD. All parameters were assessed by a paired Wilcoxon sign-rank test and determined to be significant when p<0.05.

Results

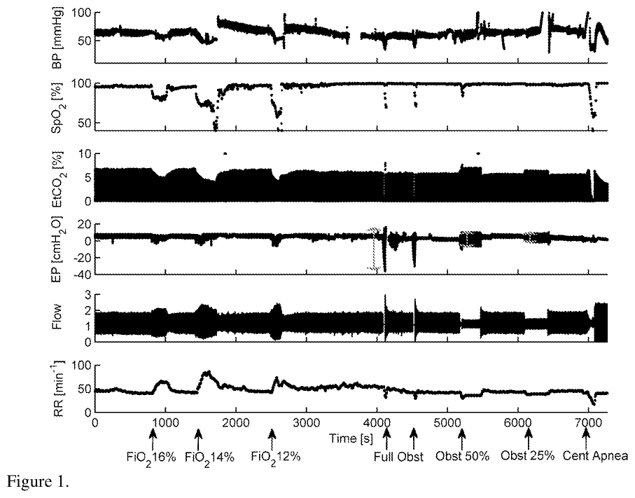

The rabbits (n=6) weighed 3.79±0.18 kg. FIG. 1 presents the BP, SpO₂, EtCO₂, EP, endotracheal flow, and respiratory rate (RR) from one experiment. The experiment was comprised of eight distinct events: three levels of hypoxia with FiO₂ of 16%, 14%, and 12%, two successive short full obstructions, two partial obstructions of 50% and 25%, and finally a central-type apnea. As expected, the SpO₂ decreased severely during hypoxia in parallel with the decrease in the EtCO₂, yielding a mirror image with the compensatory increase in the RR and the endotracheal flow. In contrast, partial obstructions were associated with a decrease in the respiratory rate. Interestingly, during both partial obstructions, SpO₂ remained practically unchanged from baseline despite the obvious increases in EtCO₂ and EP.

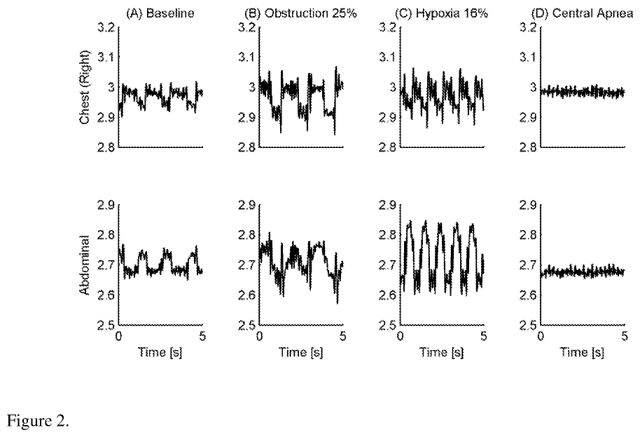

FIG. 2 depicts the raw motion signals sensed from the chest and abdomen during four event types, within a five second window. When comparing to baseline (FIG. 2A) it is evident that the amplitude of the signals during both partial obstruction (FIG. 2B) and hypoxia (FIG. 2C) increased, while it diminished during central-type apnea (FIG. 2D). Partial obstruction led to a decrease in respiratory rate, whereas an increase in respiratory rate was seen during hypoxia. Intriguingly, the shape of the breath changed significantly and exhibited sharp transitions during the partial obstruction (FIG. 2B), but changed little during hypoxia (FIG. 2C).

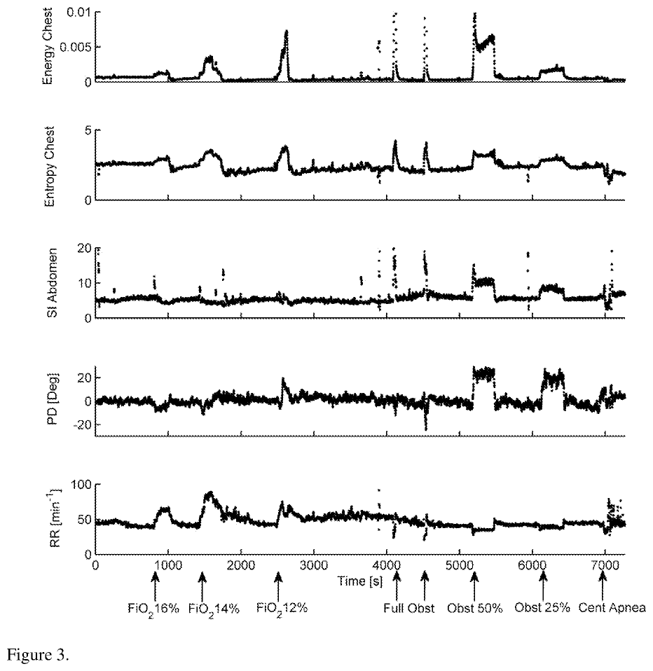

FIG. 3 presents the respiratory dynamics parameters collected throughout one experiment. Both partial obstruction and hypoxia induced an increase in energy and entropy, while central-type apnea resulted in a decrease in both parameters. Both energy and entropy had a similar morphological response to all the imposed events. The SI, PD, and the RR (lower three plots) exhibited different and even opposite responses during partial obstruction and hypoxia. Both SI and PD increased during obstructive events and remained practically unchanged during the first two hypoxic events. RR increased during hypoxic events and decreased during partial or full obstruction.

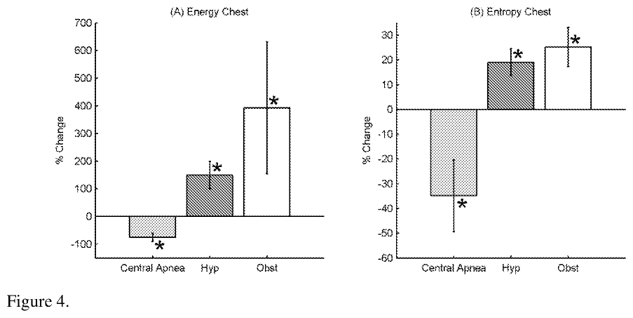

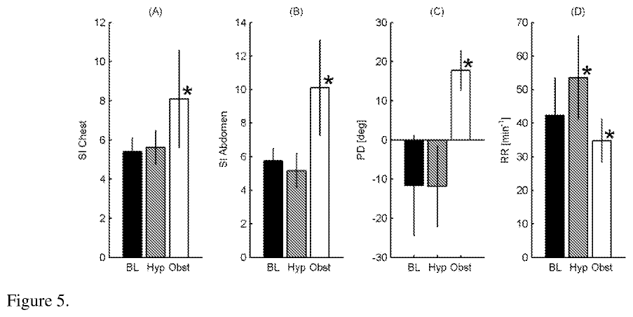

The feasibility of detecting and classifying the least severe obstruction (25%) and hypoxic (16%) events was analyzed (Tables 1 and 2). The more severe perturbations (50% obstruction and 14% hypoxia) were easier to identify and discriminate, as is evident in FIG. 3. The changes of the extracted parameters during pseudo-central apnea, 25% partial obstruction and 16% hypoxia, from all of the experiments, are summarized in FIG. 4 and FIG. 5. On average, energy increased by 150% and 400% during hypoxia and obstruction, respectively (FIG. 4). Entropy demonstrated a smaller, yet significant increase from baseline during both events, but with a smaller standard deviation (FIG. 4). During central hypopnea/apnea, both energy and entropy decreased significantly.

FIG. 5 presents the SI, PD and RR, in absolute numbers, for both obstruction and hypoxia. Changes from baseline in SI, PD and RR were evident and clearly differed between event types. On average, the SI increased by at least 75% during obstruction, but changes during hypoxia were, at most, 10% (FIG. 5A and FIG. 5B). The SI values at baseline and during hypoxia overlapped, within one standard deviation, rendering the changes during hypoxia insignificant (p=0.44). In the case of SI, values greater than 7 (p=0.03) may safely indicate an obstruction (FIG. 5).

On average, the PD increased from −10.0±16.1° by over 25° (p=0.0313) to +17.8±5.1° and did not change significantly (p=0.5625) during hypoxia (FIG. 5C). The RR clearly decreased during obstruction (p=0.03) and increased during hypoxia (p=0.03). Table 2 is a summary of the real values for each parameter during partial obstruction and hypoxia and their respective baseline values.

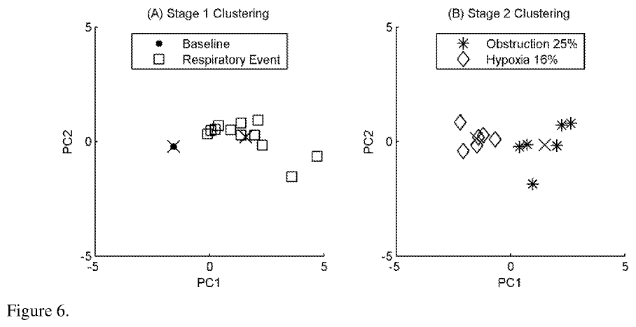

The combination of parameters improved the separation in a two stage clustering process shown in FIG. 6. In stage 1 (FIG. 6A), consideration of energy and the entropy yielded a 100% correct separation between baseline (filled dot) and obstruction or hypoxia events (unfilled square). In the second stage (FIG. 6B), the events classified as an increase in respiratory effort (i.e., obstruction, hypoxia), were clustered when the SI, chest-abdominal PD, and RR were considered. 100% correct differentiation between obstruction (star) and hypoxia (unfilled diamond) was attained (FIG. 6B).

Both the SI and PD provided good separation between obstructive and hypoxic events, individually, as depicted in the online supplementary data. The PD demonstrated a sensitivity of 83.3% and a specificity of 91.7%, the abdominal SI exhibited a sensitivity of 100% and specificity of 83.3%, and the chest SI had a sensitivity of 91.7% and specificity of 83.3%. When the SI and PD were considered together, both the sensitivity and specificity was 100%.

It is important to note that no decrease in the SpO₂ was observable even when the respiratory effort was 50% of the maximal effort. However, a mild decrease in the FiO₂ to 16% resulted in significant hypoxemia, but with no noticeable changes in the SI or the PD. Although 25% obstruction was not associated with hypoxemia, it yielded significant changes in the SI and PD indices that were not observed with mild hypoxemia (FiO₂=16%).

Discussion

Monitoring the dynamics of the chest and abdomen via miniature accelerometers, has been shown here to sense unique respiratory dynamics parameters that are sensitive to small changes in the respiratory effort and that can differentiate between central hypopnea/apnea and increase in respiratory effort. Moreover, these parameters can highly specifically differentiate between mechanical (e.g. obstruction) and non-mechanical (e.g. hypoxia) causes of increased respiratory effort. The four distinct indices of energy, entropy, shape-index, and phase-difference, are indicative of a gamut of important characteristics during breathing: amplitude, breath-to-breath changes, shape of breathing motion, and the synchrony between the chest and abdomen. Breath energy and entropy were shown to effectively reflect increases (obstruction and hypoxia) or decrease (central hypopnea\apnea) in respiratory effort. The novel indices, shape-index and phase-difference, are instrumental in providing the necessary specificity to discern between mechanical and non-mechanical causes of increased respiratory effort.

Increased respiratory effort also occurs in the presence of normal airway resistance and lung compliance. This non-obstructive group includes physiological response to hypoxia, the active phase of sleep in preterm and term infants [7, 26, 27], and compensatory responses to an increase in oxygen consumption. Therefore, the study sought out a means of differentiating between partial obstructions and respiratory events induced by non-mechanical modifications (hypoxia), based on the changes in ventilation dynamics.

By implementing a two-stage classification technique, it was possible to effectively identify and classify events either as one with increased respiratory effort or as central hypopnea/apnea, and thereafter to characterize the increase in respiratory effort events as either partially obstructive or non-obstructive\hypoxic events.

Breath energy and entropy indices can identify and classify events of increased respiratory effort and central apnea, with a sensitivity of 100%, making them appropriate parameters for implementation in the first stage of classification. The SI and PD indices are instrumental and appropriate for the second stage of classification. Both indices are substantially higher during partial obstruction and both remain unchanged during hypoxia. The SI, which quantifies breathing waveform complexity, is low for smooth semi-sinusoidal respiratory waves and high for sharp respiratory waves with abrupt changes in the respiratory dynamics and polyphasic structure, as occurs in flattening airflow waveforms that are characteristics to high resistance in the airway. The SI is only sensitive to changes in wave shape and is independent of the amplitude or the duration of the breath. Interestingly, the observations presented here imply that an SI exceeding a value of 7 can be considered an obstructive event, and that an absolute threshold can be defined for identifying obstructive event. Obviously, this is an observation in a preclinical study and must be confirmed in extensive clinical studies.

The PD index is also highly specific to obstructive events; it is negative at baseline and remains unchanged during hypoxic events. In contrast, during obstruction, it undergoes a profound change, resulting in a positive phase difference. Interestingly, our findings imply that a PD larger than 10° is indicative of an obstruction. Therefore, an absolute threshold for PD can also be defined for identification of obstructive events. Determining a phase relation between the chest and abdomen based on the volume of the chest and abdomen has typically been implemented in plethysmographic studies [11,12,17,28,29]. However, the methods used to date, rely on clear sinusoidal waveforms, with a clear time shift between the chest and abdomen. These indices of thoracoabdominal asynchrony have proven insufficient and non-specific (specificity of 10.9%) in detecting obstructive apnea [12]. Moreover, non-sinusoidal signals observed during obstruction, with flattened airflow, lead to erroneous measurements of these indices [11]. The present method, utilizing the Hilbert transform, loosens the constraint on the morphology of the signal, rendering it much less susceptible to noise, and more accurate in phase calculations (See online supplement).

RR increased during hypoxia and decreased during an obstruction. The decrease in the RR during obstruction can be explained by the need for a more prolonged increase in respiratory effort, due to the flattening of the flow waveform. This observation is congruent with the reported increase in inspiratory time during obstructive episodes [30]. However, the RR was less effective in separating between modes of increased respiratory effort (Fig E1 in the online supplement), when compared to the SI and the PD, and is not crucial for separation of groups. Both the SI and PD provided good separation individually. When the SI and PD were applied together to identify respiratory events, the sensitivity and specificity were 100% (no false positive or false negative differentiation of partial obstructive from hypoxic and central hypopneic events).

The EP serves as the gold standard for monitoring increases in the respiratory effort and for detection of obstruction, however, measurement is invasive, inconvenient and poorly tolerated in adults and is rarely used in infants [8]. Our previous study focused on fast detection of full obstructive apnea and compared this method to EP [24]. Here the EP was used to define the severity of the partial obstruction (25% or 50%), and the increases in the energy and entropy indices correspond to the severity of the obstruction defined by the EP. The amplitude of the respiratory effort is assessed by the EP, tidal breath displacement [24], energy or entropy. The novel PI and PD indices provide additional essential information that enable to differentiate between obstructive and non-obstructive increase in the effort, since the SI and PD indices are independent of the breath signal amplitudes, and are sensitive to changes in the shapes and phases of the signals.

Conclusions

A simple non-invasive modality that utilizes three miniature sensors (<1 g) provides a gamut of indices that enable the identification and classification of partial obstructions and hypopneic/apneic events. The SI and PD indices are effective in capturing the changes in breath waveforms and inherent chest-abdominal phase relation, and are the most specific in identifying obstructive events. Its applicability in preventing ASSB in infants and improving the accuracy of partial obstruction detection in children and adults should be further investigated.

Tables

TABLE 1
The significances of the changes (P-Values) in the
various parameters relative to the baseline values.

| | Central-type | Obst. | Hyp. |
|---|---|---|---|
| Parameters | Apnea | 25% | 16% |
| Energy Chest | 0.03 | 0.03 | 0.03 |
| Entropy Chest | 0.03 | 0.03 | 0.03 |
| Shape Index | — | 0.03 | 0.16 |
| Phase Difference | — | 0.03 | 0.56 |
| Respiratory Rate | — | 0.03 | 0.03 |

TABLE 2
The values of the respiratory dynamics parameters
during baseline (just prior to perturbations), mild obstruction
(25%) and mild hypoxia (FiO₂ = 16%).

| Parameter | BL | Obst | BL | Hyp |
|---|---|---|---|---|
| Enerey | 0.0004 | 0.0017 | 0.0004 | 0.0010 |
| Entropy | 2.278± | 2.787± | 2.323± | 2.795± |
| Shape | 5.793± | 10.102 | 5.762± | 5.179± |
| Phase | −10.025 | 17.778 | −11.655 | −11.832 |
| Respirator | 43.733 | 34.805 | 42.267 | 53.576 |

### REFERENCES

1. Shapiro-Mendoza C K, Kimball M, Tomashek K M, Anderson R N, Blanding S. US Infant Mortality Trends Attributable to Accidental Suffocation and Strangulation in Bed From 1984 Through 2004: Are Rates Increasing? PEDIATRICS 2009; 123: 533-539.

2. Task Force on Sudden Infant Death Syndrome. SIDS and Other Sleep-Related Infant Deaths: Expansion of Recommendations for a Safe Infant Sleeping Environment. PEDIATRICS 2011; 128: e1341-e1367.

3. Kato I, Groswasser J, Franco P, Scaillet S, Kelmanson I, Togari H, Kahn A. Developmental characteristics of apnea in infants who succumb to sudden infant death syndrome. Am. J. Respir. Crit. Care Med. 2001; 164: 1464-1469.

4. Kinney H C, Thach B T. The sudden infant death syndrome. N. Engl. J. Med. 2009; 361: 795-805.

5. Ramanathan R, Corwin M J, Hunt C E, Lister G, Tinsley L R, Baird T, Silvestri J M, Crowell D H, Hufford D, Martin R J, Neuman M R, Weese-Mayer D E, Cupples L A, Peucker M, Willinger M, Keens T G, Collaborative Home Infant Monitoring Evaluation (CHIME) Study Group. Cardiorespiratory events recorded on home monitors: Comparison of healthy infants with those at increased risk for SIDS. JAMA 2001; 285: 2199-2207.

6. Katz E S, Mitchell R B, D'Ambrosio C M. Obstructive Sleep Apnea in Infants. Am. J. Respir. Crit. Care Med. 2012; 185: 805-816.

7. Marcus C L. Sleep-disordered breathing in children. Am. J. Respir. Crit. Care Med. 2001; 164: 16-30.

8. Argod J, Pepin J L, Smith R P, Levy P. Comparison of esophageal pressure with pulse transit time as a measure of respiratory effort for scoring obstructive nonapneic respiratory events. Am. J. Respir. Crit. Care Med. 2000; 162: 87-93.

9. Al-Angari H M, Sahakian A V. Automated Recognition of Obstructive Sleep Apnea Syndrome Using Support Vector Machine Classifier. Information Technology in Biomedicine, IEEE Transactions on 2012; 16: 463-468.

10. Morillo D, Ojeda J, Foix L, Jimenez A. An Accelerometer-Based Device for Sleep Apnea Screening. Information Technology in Biomedicine, IEEE Transactions on 2010; 14: 491-499.

11. Hammer J, Newth C J L. Assessment of thoraco-abdominal asynchrony. Paediatr Respir Rev 2009; 10: 75-80.

12. De Groote A, Groswasser J, Bersini H, Mathys P, Kahn A. Detection of obstructive apnea events in sleeping infants from thoracoabdominal movements. J Sleep Res 2002; 11: 161-168.

13. Warren R H, Horan S M, Robertson P K. Chest wall motion in preterm infants using respiratory inductive plethysmography. Eur Respir J 1997; 10: 2295-2300.

14. Varady P, Bongar S, Benyo Z. Detection of airway obstructions and sleep apnea by analyzing the phase relation of respiration movement signals. IEEE Trans. Instrum. Meas. IEEE; 2003; 52: 2-6.

15. Williamson J R, Bliss D W, Browne D W, Indic P, Bloch-Salisbury E, Paydarfar D. Individualized apnea prediction in preterm infants using cardio-respiratory and movement signals. BSN 2013; :1-6.

16. Leder R S, Cohen K P, Peterson N, Palreddy S, Green C, Tompkins W J, Webster J G. Performance of impedance and inductance ventilation sensors on premature infants during natural breathing, motion, and airway obstruction. IEEE; 2003; 4: 3094-3097.

17. Weese-Mayer D E, Corwin M J, Peucker M R, Di Fiore J M, Hufford D R, Tinsley L R, Neuman M R, Martin R J, Brooks L J, Davidson Ward S L, Lister G, Willinger M. Comparison of apnea identified by respiratory inductance plethysmography with that detected by end-tidal CO(2) or thermistor. The CHIME Study Group. Am. J. Respir. Crit. Care Med. 2000; 162: 471-480.

18. Azarbarzin A, Moussavi Z M K. Automatic and unsupervised snore sound extraction from respiratory sound signals. IEEE Trans Biomed Eng 2011; 58: 1156-1162.

19. Montazeri A, Giannouli E, Moussavi Z. Assessment of Obstructive Sleep Apnea and its Severity during Wakefulness. Ann Biomed Eng 2011.

20. Ben-Israel N, Tarasiuk A, Zigel Y. Obstructive apnea hypopnea index estimation by analysis of nocturnal snoring signals in adults. Sleep 2012; 35: 1299-305C.

21. Pesin J, Faingersh A, Waisman D, Landesberg A. Highly sensitive monitoring of chest wall dynamics and acoustics provides diverse valuable information for evaluating ventilation and diagnosing pneumothorax. Journal of Applied Physiology 2014; 116: 1632-1640.

22. Waisman D, Levy C, Faingersh A, Klotzman F I C, Konyukhov E, Kessel I, Rotschild A, Landesberg A. A new method for continuous monitoring of the chest wall movement to characterize hypoxemic episodes during HFOV. Intensive Care Medicine 2011; 37(7): 1174-1181.

23. De Abreu M G, Guldner A. Early detection of deteriorating ventilation: prevention is better than cure! Intensive Care Med 2012; 38(1): 7-8

24. Waisman D, Lev-Tov L, Levy C, Faingersh A, Klotzman F I C, Bibi H, Rotschild A, Landesberg A. Real-time detection, classification and quantification of apneic episodes using miniature surface motion sensors in rats. Pediatric Research 2015; 78:63-70.

25. Chen L, Scharf S M. Comparative hemodynamic effects of periodic obstructive and simulated central apneas in sedated pigs. J Appl Physiol 1997; 83: 485-494.

26. Borghese I F, Minard K L, Thoman E B. Sleep rhythmicity in premature infants: Implications for developmental status. Sleep American Academy of Sleep Medicine; 1995.

27. Paul K, Krajča V, Roth Z, Melichar J, Petranek S. Comparison of quantitative EEG characteristics of quiet and active sleep in newborns. Sleep Medicine 2003; 4: 543-552.

28. Agostoni E, Mognoni P. Deformation of the chest wall during breathing efforts. Journal of Applied Physiology 1966; 21: 1827-1832.

29. Konno K, Mead J. Measurement of the separate volume changes of rib cage and abdomen during breathing. Journal of Applied Physiology 1967; 22: 407-422.

30. Mooney A M, Abounasr K K, Rapoport D M, Ayappa I. Relative prolongation of inspiratory time predicts high versus low resistance categorization of hypopneas. J Clin Sleep Med 2012; 8: 177-185.

What is claimed is:

1. A method for monitoring dynamics of a chest and abdomen of a patient, comprising:
using one or more sensors to sense respiratory dynamics and changes in respiratory effort;
processing breath signals from said one or more sensors to identify an increase in the respiratory effort and to identify a change in shapes of the breath signals and/or a change in chest-abdominal movement synchrony and using the increase in the respiratory effort and/or change in shapes of the breath signals and/or change in chest-abdominal movement synchrony to differentiate between central hypopnea and apnea.

2. The method according to claim 1, comprising using the change in chest-abdominal movement synchrony to differentiate between normal synchronous actuation of respiration and abnormal work against partial obstruction.

3. The method according to claim 1, comprising using the increase in the respiratory effort and the change in shapes of the breath signals and/or the change in the chest-abdominal movement synchrony to differentiate between partial upper airway obstruction and breathing without airway obstruction, wherein said increase in the respiratory effort and the change in shapes of the breath signals and/or the change in the chest-abdominal movement synchrony significantly occur during partial upper airway respiratory obstructions but do not significantly occur during breathing without airway obstruction.

4. The method according to claim 1, wherein abnormal work against partial obstruction is identified by an increase in the respiratory effort characterized by energy and entropy of the respiratory effort, and changes in shapes of the breath signals and abdominal movement synchrony.

5. The method according to claim 1, wherein the change in shapes of the breath signals comprises a change to more rectangular shaped signals.
